(19) United States
(12) Patent Application Publication
(10) Pub. No.: US 2026/0346278 A1
(43) Pub. Date: Nov. 5, 2026

(54) IMPROVED MICROFLUIDIC DEVICE

(71) Applicant: invoX Belgium NV, Diepenbeek (BE)

(72) Inventor: Frank Bartels, Hattingen (DE)

(21) Appl. No.: 19/166,067

(22) PCT Filed: Mar. 15, 2024
(86) PCT No.: PCT/EP2024/057014

§ 371 Date: Sep. 17, 2025

(30) Foreign Application Priority Data
Mar. 17, 2023 (EP) 23162506.2

(51) Int. Cl. B05B 1/26; A61M 11/00; A61M 15/00

(52) U.S. Cl. CPC B05B 1/262; A61M 11/003; A61M 15/009

(57) ABSTRACT

The invention provides a nozzle assembly (1) for the nebulization of a liquid, preferably of a medically active liquid, the nozzle assembly (1) comprising a first member (2) comprising a first surface (4) and a second member (3) comprising a second surface, wherein the first member (2) comprises at least one positioning element (6) integrally formed on the first member (2), and wherein in the assembled state of the nozzle assembly (1)—the first surface (4) contacts the second surface, thereby defining a contact plane,—the nozzle assembly (1) comprises a fluid inlet (7), a fluid outlet (8) and a connecting structure (9), all of which are in fluid communication forming a channel for the transport of the liquid in a downstream direction from the fluid inlet (7) via the connecting structure (9) to the fluid outlet (8),—said channel is in contact with the contact plane and -the at least one positioning element (6) formed on the first member (2) is in physical interaction with the second member (3) thereby preventing a displacement of the first member (2) relative to the second member (3).

### BACKGROUND OF THE INVENTION

The present invention is in the field of nozzle assemblies, specifically in the field of nozzle assemblies for medical devices.

### SUMMARY OF THE INVENTION

In a first aspect, the invention relates to a nozzle assembly (1) for the nebulization of a liquid, preferably of a medically active liquid, the nozzle assembly (1) comprising a first member (2) comprising a first surface (4) and a second member (3) comprising a second surface (5), wherein the first member (2) comprises at least one positioning element (6) integrally formed on the first member (2), and
wherein in the assembled state of the nozzle assembly (1)
the first surface (4) contacts the second surface (5), thereby defining a contact plane,
the nozzle assembly (1) comprises a fluid inlet (7), a fluid outlet (8) and a connecting structure (9), all of which are in fluid communication forming a channel for the transport of the liquid in a downstream direction from the fluid inlet (7) via the connecting structure (9) to the fluid outlet (8),
said channel is in contact with the contact plane, and
the at least one positioning element (6) formed on the first member (2) is in physical interaction with the second member (3) thereby preventing a displacement of the first member (2) relative to the second member (3).

In one embodiment of this aspect the first and/or second member (3) are made from a crystalline material or glass. In a further embodiment the connecting structure (9) comprises at least one microfluidic element, wherein the at least one microfluidic element comprises a filter structure comprising at least one filter element (10). In some embodiments the nozzle assembly (1) comprises both features.

In a second aspect, the invention relates to the use of said nozzle assembly (1) in a device for nebulizing a liquid.

In a further aspect, the present invention provides a nebulization device comprising a nozzle assembly (1) as defined above.

### BRIEF DESCRIPTION OF THE DRAWINGS

FIGS. 1A and 1B depict a simple nozzle assembly (1) according to the prior art.

FIGS. 2A to 2D show a nozzle assembly (1) comprising positioning elements (6) according to the present invention.

FIGS. 3A to 3C show specific embodiments of the invention utilizing a specific type of positioning element (6).

FIGS. 4A and 4B show different possible variants of positioning elements (6).

FIGS. 5A and 5B show an embodiment, which utilizes microfluidic elements on both members of the nozzle assembly (1).

FIGS. 6A and 6B show an embodiment of a nozzle assembly (1) according to the present invention comprising filter elements (10).

### DETAILED DESCRIPTION OF THE INVENTION

The present invention relates to a novel nozzle assembly (1) for the nebulization of liquids. The assembly comprises two members, a first and a second member (3) which comprise the microfluidic elements of the nozzle, including the fluid inlet (7) and outlet (8). In the assembled state of the nozzle assembly (1), the two members are in contact with each other, forming a contact plane, thereby creating a nozzle with a fluid inlet (7) and outlet that are fluidically connected to each other.

The two members of the nozzle assembly (1) comprise at least one positioning element (6), which allows for a precise combination of the two members and preventing a displacement of the two members relative to each other. This allows for a more precise positioning of microfluidic elements and allows for microfluidic elements on both members to improve the nozzle assembly (1).

A major advantage of the nozzle assembly (1) according to the present invention is that the claimed assembly reduces manufacturing errors, which in turn improves the overall quality of the nozzle assembly (1) and allows for a higher flexibility on the production of the nozzle assembly (1).

Accordingly, in a first aspect, the present invention relates to a nozzle assembly (1) for the nebulization of a liquid, preferably of a medically active liquid, the nozzle assembly (1) comprising a first member (2) comprising a first surface (4) and a second member (3) comprising a second surface (5), wherein the first member (2) comprises at least one positioning element (6) integrally formed on the first member (2), and
wherein in the assembled state of the nozzle assembly (1)
the first surface (4) contacts the second surface (5), thereby defining a contact plane,
the nozzle assembly (1) comprises a fluid inlet (7), a fluid outlet (8) and a connecting structure (9), all of which are in fluid communication forming a channel for the transport of the liquid in a downstream direction from the fluid inlet (7) via the connecting structure (9) to the fluid outlet (8),
said channel is in contact with the contact plane, and
the at least one positioning element (6) formed on the first member (2) is in physical interaction with the second member (3) thereby preventing a displacement of the first member (2) relative to the second member (3).

An advantage of the presence of the positioning element (6) on the first member (2), interacting with the second member (3) is that it allows for easier positioning with a higher precision. This allows for improved nozzle assemblies, in particular in a case where both members comprise specific structures that interact with each other. Such structures are in particular microfluidic structures. In these embodiments, the two members can be combined with a higher precision and allow for particular advantages that would be impossible if only one member comprised the structures.

As such, the invention relates to two particularly preferred embodiments, which may be combined in a further embodiment.

In the first preferred embodiment, the invention relates to a nozzle assembly (1) as defined above, wherein the first and/or second member (3) are made from a rigid non-plastic material, such as a crystalline material or glass.

In the second preferred embodiment, the invention relates to a nozzle assembly (1), wherein in the assembled state of the nozzle assembly (1) the connecting structure (9) comprises at least one microfluidic element, wherein the at least one microfluidic element comprises a filter structure comprising at least one filter element (10). The filter structure may be formed by structures on both the first and second member (3). An advantage of said embodiment is that the filter structure allows for a higher filter resolution than a filter structure on a single member.

In a further embodiment of the invention, the invention relates to a nozzle assembly (1) as defined above, wherein the first and/or second member (3) are made from a rigid non-plastic material, such as a crystalline material or glass, and wherein in the assembled state of the nozzle assembly (1) the connecting structure (9) comprises at least one microfluidic element, wherein the at least one microfluidic element comprises a filter structure comprising at least one filter element (10).

The different embodiments will be explained in more detail below.

In general, the nozzle assembly (1) is a nozzle assembly (1) for nebulizing a liquid. The nozzle assembly (1) may be used with any suitable nebulizer. As such, the nozzle assembly (1) may comprise one or more fluid outlets (8), depending on the type of nebulizer. In preferred embodiments the nozzle assembly (1) comprises a plurality of fluid outlets (8).

The first and second member (3) each comprise a first and second surface (5) respectively. Said first and second surface (5) are preferably planar surfaces and said contact plane essentially corresponds to the first and/or second surface (5).

The fluid outlets (8) may be formed on one of the two members of the nozzle assembly (1) or may be formed on both members. In some embodiments the fluid outlet (8) is formed on one member only. In some embodiments, corresponding structures on both members form the fluid outlet (8). In the case of a plurality of fluid outlets (8), each outlet may be formed on the same member, or is formed by corresponding structures on both members. In some embodiments, in the assembled state the nozzle assembly (1) comprises a plurality of fluid outlets (8) wherein for each outlet individually the outlet is formed solely on one member or formed by both members.

The nozzle assembly (1) comprises in the assembled state a fluid inlet (7), a fluid outlet (8) and a connecting structure (9), all of which are in fluid communication forming a channel for the transport of the liquid in a downstream direction from the fluid inlet (7) via the connecting structure (9) to the fluid outlet (8). Said fluid inlet (7), fluid outlet (8) and connecting structure (9) may be present on one of the first and second member (3) or may be distributed over both members.

In some embodiments of the invention, the liquid inlet, liquid outlet and the connecting structure (9) are provided on the first member (2). In these embodiments, the second member (3) may or may not comprise additional microfluidic structures, which may interact with microfluidic structures on the first member (2).

In other embodiments, the liquid inlet, liquid outlet and the connecting structure (9) are provided on the second member (3). In these embodiments, the first member (2) may or may not comprise additional microfluidic structures, which may interact with microfluidic structures on the second member (3).

In some embodiments, the at least one of the fluid inlet (7), fluid outlet (8) and connecting structure (9) are provided in the first and second member (3), preferably in the form of a channel.

Accordingly, in some embodiments the invention relates to a nozzle assembly (1) as described above, wherein the at least one of the fluid inlet (7), fluid outlet (8) and connecting structure (9) which is provided in the form of a channel formed in at least one of the first surface (4) and the second surface (5) has a depth of from about 1 μm to about 100 μm, preferably of from about 3 μm to about 30 μm.

In some embodiments of the invention, none of the fluid inlet (7), the fluid outlet (8) and the connecting structure (9) are provided on the second surface.

In a further embodiment, the invention relates to a nozzle assembly (1) as defined above, wherein the fluid inlet (7), the fluid outlet (8) and the connecting structure (9) are located on the first surface (4) of the first member (2) and on the second surface (5) of the second member (3). In particular embodiments the fluid inlet (7), the fluid outlet (8) and the connecting structure (9) are provided in the form of mirror-image structures on the first and the second surface (5).

In some embodiments of the invention, the connecting structure (9) provided on at least one of the first and second surface (5) comprises bottom and lateral walls connecting the bottom with the first or the second surface (5). In specific embodiments, said lateral walls are angled such that the inner diameter of the at least one of the fluid inlet (7), fluid outlet (8) and microfluidic structure widens from the bottom towards the first or second surface (5), correspondingly.

In a further aspect of the invention, at least one of the fluid inlet (7), the fluid outlet (8) and the connecting structure (9) are provided on the second member (3) with the other structures present on the first member (2).

In particular embodiments of the invention, at least one of the liquid inlet and liquid outlet is provided on the first member (2) and the connecting structure (9) is provided on the second member (3). In some embodiments, parts of the connecting structure (9) are provided on the first and second member (3).

The nozzle assembly (1) utilizes positioning elements (6), which are integrally formed on one of the two members of the nozzle assembly (1), which interact with the other member. This allows for a higher precision of the positioning of the two members and prevents a displacement. This can be improved by usage of a plurality of positioning elements (6). Said plurality of positioning elements (6) may be integrally formed on one member or on both members.

Accordingly, in one embodiment of the invention, the second member (3) also comprises at least one positioning element (6) integrally formed on the second member (3), wherein in the assembled state of the nozzle assembly (1) the at least one positioning element (6) formed on the second member (3) is in physical interaction with the first member (2), thereby preventing a displacement of the first member (2) relative to the second member (3).

In further embodiments of the invention, the first member (2) comprises a plurality of positioning elements (6). In some embodiments, the first and second member (3) comprise a plurality of positioning elements (6).

The at least one positioning element (6) or the plurality of positioning elements (6) can have any suitable form to prevent a displacement. The skilled person is aware of suitable positioning elements (6). Sad positioning elements (6) may be placed in different positions and can have different shapes.

As such, in some embodiments of the invention, the positioning element (6) formed on the first member (2) is located on the first surface (4). In some embodiments, the nozzle assembly (1) comprises a plurality of positioning elements (6) formed on the first member (2), located on the first surface (4).

In further embodiments, the nozzle assembly (1) comprises a positioning element (6) formed on the second member (3), wherein said positioning element (6) formed on the second member (3) is located on the second surface (5).

The positioning element (6) may be in the form of a protrusion or indentation. The positioning element (6) may be of any shape. In some embodiments, the nozzle assembly (1) comprises complementary positioning elements (6) on the first and second member (3).

In some embodiments of the invention, the positioning element (6) formed on the first member (2) is provided in the form of a projection (protuberance) projecting from the first surface (4). Said projection may be in the form of a pillar, for example a round or rectangular pillar. The projection may be of irregular shape, e.g. a star or similar.

In some embodiments, in the assembled state of the nozzle assembly (1), the projection is projecting from the first surface (4) and extends beyond the contact plane of the nozzle assembly (1). In some embodiments the second member (3) is smaller than the first member (2) and the projections are able to hold the second member (3) in place. In some embodiments the projection on the first member (2) is a circumferential wall around the borders of the first member (2)s, holding the second member (3) inside. Said wall may fully or partly enclose the second member. The second member (3) therefore may be of the same size or a different size than the first member (2).

In some embodiments, the nozzle assembly (1) comprises complementary positioning elements (6), such as a projection and an indentation in complementary positions on the first and second member (3).

Accordingly, in some embodiments, a positioning element (6) in the nozzle assembly (1) is an indentation or recess. Said indentation may be on a complementary position to a projection on the other member.

In particular, in some embodiments, the invention relates to a nozzle assembly (1) comprising a positioning element (6) on the first member (2) and a positioning element (6), wherein the positioning element (6) formed on the second member (3) is provided in the form of an indentation (recess) formed in the second surface (5).

In case of a plurality of positioning elements (6), the first and/or second member (3) may comprise different types of positioning elements (6). For example, one member may comprise a projection and an indentation.

Accordingly, in one embodiment of the invention, the plurality of positioning elements (6) formed on the first member (2) comprises a positioning element (6) in the form of a projection projecting from the first surface (4) and a positioning element (6) in the form of an indentation (recess) formed in the first surface (4).

In some embodiments, the different positioning elements (6) are found on the second member (3). Accordingly, in some embodiments, the invention relates to a nozzle assembly (1) as defined above, comprising a plurality of positioning elements (6) on the second member (3), wherein the plurality of positioning elements (6) formed on the second member (3) comprises one projection projecting from the second surface (5) and an indentation (recess) formed in the second surface (5).

Said projection and indentation may take any suitable shape. In particular, said projection and indentation may have the shape of a pin and a corresponding hole.

Accordingly, in some embodiments, the invention relates to a nozzle assembly (1) as defined above, wherein the at least one positioning element (6) formed on the first member (2) in the form of a projection (protuberance) projecting from the first or second surface (5) is provided in the form of a pin.

In some embodiments, the invention relates to a nozzle assembly (1) as defined above, wherein the at least one positioning element (6) formed on the first or second member (3) in the form of an indentation (recess) formed in the second surface (5) is provided in the form of a hole.

In some embodiments, the positioning elements (6) have specific cross-sectional shapes, such as like a star or a rectangle. Preferably, the cross-sectional shape of a positioning element (6) on one member corresponds to the cross-sectional shape of a complementary positioning element (6) on the other member. Said corresponding positional elements are in the same position on the contact plane.

In some embodiments, wherein the positioning element (6) is provided in form of a protrusion, said protrusion is provided in a form of a linear or closed loop protrusion. In some embodiments, said protrusion is a linear protrusion wherein the linear protrusion is in the form of a straight or curved line or pattern of a plurality of linear protrusions.

In some embodiments, said protrusion is in the form of a closed loop protrusion, wherein the closed loop protrusion is provided in the form of a circular, oval, polygonal, star shaped or irregular shaped closed loop protrusion.

In preferred embodiments, at least one positioning element (6) formed on the first or second member (3) is in the form of a projection or protuberance projecting from the first or second surface (5) and is provided in the form of a pin and at least one positioning element (6) formed on the first or second member (3) is in the form of an indentation or recess formed in the second surface (5) and is provided in the form of a hole. In particular preferred embodiments of the invention said positioning members are in the same position on the contact plane.

Accordingly, in some embodiments. the invention relates to a nozzle assembly (1) as defined above, wherein the cross-sectional shape of the at least one positioning element (6) provided on the first surface (4) corresponds to the cross-sectional shape of the at least one positioning element (6) provided on the second surface (5).

The positioning elements (6) may be in the form of projections and indentations, as defined above with different cross-sectional shapes. The positional elements may have a suitable height or depth. Preferably, the at least one positioning element (6), when provided in the form of a projection, has a height of from about 1 to about 100 μm, preferably of from about 3 to 30 μm as measured from the corresponding first or second surface (5). Analogously, it is preferred that the at least one positioning element (6), when provided in the form of an indentation, has a depth of from about 1 to about 100 μm, preferably of from about 3 to 30 μm as measured from the corresponding first or second surface (5).

The positioning element (6) prevents the displacement of the two members relative to each other. The prevented displacement may be horizontally or vertically. Accordingly, in one embodiment. the invention relates to a nozzle assembly (1) according as defined above, wherein the at least one positioning element (6) is adapted to prevent a horizontal displacement of the first member (2) relative to the second member (3) within the contact plane.

In some embodiments, the at least one positioning element (6) is adapted to prevent vertical displacement of the first member (2) relative to the second member (3) relative to the contact plane. In some embodiments, the at least one positioning element (6) is adapted to prevent horizontal and vertical displacement of the first member (2) relative to the second member (3) relative to the contact plane.

The positioning element (6) of the first member (2) is in physical interaction with the second member (3). Preferably, said physical interaction is a connection of the two members in a way as described above. Said connection may be reversible or irreversible.

In some embodiments, the invention relates to a nozzle assembly (1), wherein in the assembled state the at least one positioning element (6) is adapted to generate a firm (preferably irreversible) connection between first member (2) and second member (3).

In some embodiments, the invention relates to a nozzle assembly (1), wherein in the assembled state the at least one positioning element (6) is adapted to generate a reversible connection between first member (2) and second member (3).

The positioning elements (6) may be placed anywhere on the respective member. In particular said positioning elements (6) may or may not be in contact with the liquid to be nebulized by the nozzle assembly (1).

Accordingly, in some embodiments, the invention relates to a nozzle assembly (1) as defined above, wherein in the assembled state of the nozzle assembly (1) the at least one positioning element (6) is not in contact with the liquid to be nebulized.

In further embodiments, the invention relates to a nozzle assembly (1) as defined above, wherein in the assembled state of the nozzle assembly (1) the at least one positioning element (6) contacts the liquid to be nebulized.

As noted before in case of a plurality of positioning elements (6), the nozzle assembly (1) may comprise positioning elements (6) in both members which are complementary to each other and in complementary positions. As such, in some embodiments, the positioning element (6) or the plurality of positioning elements (6) formed on the first surface (4) has the same position as the corresponding positioning element (6) or the plurality of positioning elements (6) formed on the second surface (5) with regard to the contact plane.

In particular embodiments, the positioning elements (6) on one of the members mesh with the positioning elements (6) on the other member. Accordingly, in one embodiment, the positioning element (6) or the plurality of positioning elements (6) formed on the first surface (4) mesh with the positioning element (6), or the plurality of positioning elements (6) formed on the second surface (5) in the assembled state of the nozzle assembly (1).

The nozzle assembly (1) may only require one single positioning element (6) on one element to prevent displacement of the two members relative to each other. The number of positioning elements (6) in the nozzle assembly (1) can depend on the type and form of the positioning element (6). In some embodiments, one positioning element (6) on each member is sufficient, for example if the positioning elements (6) are form fitting meshing positioning elements (6). In other embodiments, two positioning elements (6) on each member can be required. In certain embodiments a single positioning element (6) is needed.

In some embodiments, the connecting structure (9) comprises at least one microfluidic element. Such microfluidic elements may include microfluidic channels or a microfluidic filter element (10). In these embodiments, it is preferred that at least one microfluidic element is present on each member. Said microfluidic elements include the fluid inlet (7), the fluid outlet (8) and the connecting structure (9) as well as microfluidic structures realized therein, such as a filter structure.

In the context of the present invention, the term “connecting structure” refers to any structure and microfluidic element therein, which is within the fluid connection between the fluid inlet (7) and fluid outlet (8). In the simplest form of the nozzle assembly (1), the connecting structure (9) is a channel from the fluid inlet (7) to the fluid outlet (8) or outlets. However, the connecting structure (9) may comprise additional microfluidic structures.

According in some embodiments the invention relates to a nozzle assembly (1) as defined above, wherein the connecting structure (9) comprises at least one microfluidic element provided on the second surface (5) of the second member (3). Said microfluidic elements can be introduced into the respective members by any suitable technique. Said techniques include, but are not limited to etching, scratching, embossing and/or cutting.

Accordingly, in some embodiments the invention relates to a nozzle assembly (1) as defined above, comprising at least one microfluidic element, wherein the at least one microfluidic element and optionally at least one of the fluid inlet (7) and the fluid outlet (8) are provided by etching, embossing, scratching and/or cutting the corresponding element into the first and/or second surface (5) of the first and/or second member (3).

In accordance with the first preferred embodiment, the present invention is particularly suitable if at least part of the nozzle assembly (1) is made from a non-plastic material. Non-plastic materials suitable for the present invention include crystalline materials, metal, glass, and/or inorganic polymeric materials. Preferred materials are crystalline materials and glass.

The nozzle assembly (1) may be made from different materials. In some embodiments one of the two members may be made from a non-plastic material, in preferred embodiments both, the first and second member (3), are made from a non-plastic material. Even if the first and/or second member (3) is made from plastic, it is preferred that the first and/or second member (3) are not obtained by injection moulding.

The first and second member (3) may be made from different materials or the same material. It is preferred that at least one member is made from a non-plastic material. In some embodiments the first and second member (3) are made of the same material.

The skilled person is aware of suitable non-plastic materials. Suitable materials include, but are not limited to, glass, metals, e.g. iron, steel, aluminium, germanium, copper, silver, gold, or crystalline materials, such as silicone or gallium arsenide.

In case of crystalline materials, the material may be a mono-crystalline material, preferably a mono-crystalline material crystallizing in the diamond structure or perovskite-structure, such as mono-crystalline silicone, mono-crystalline germanium, or mono-crystalline gallium arsenide. In a preferred embodiment the first and/or second member (3) are made from mono-crystalline silicone. The first and/or second member (3) may be obtained from a mono-crystalline wafer.

As such, in some embodiments, the invention relates to a nozzle assembly (1) as defined above, wherein at least one of the first and second member (3) comprises a crystalline material, in some embodiments a mono-crystalline material. In some embodiments, the first and the second member (3) each comprise a crystalline material, preferably a mono-crystalline material. The crystalline material of the first and second member (3) may be the same or a different material.

In particular embodiments of the invention one of the first and second member (3)s comprises a crystalline material, preferably a mono-crystalline material, and the other one of the first and second member (3)s comprises glass.

In a particular aspect, the invention relates to a nozzle assembly (1) as defined above, wherein the at least one microfluidic element comprises a filter structure comprising at least one filter element. Said filter structure may be of any suitable filter structure. Suitable structures are known to the skilled person and include for example pillar structures that may act as a filter structure.

In a preferred embodiment, the invention relates to a nozzle assembly (1) comprising at least one microfluidic element, wherein said microfluidic element comprises a filter structure, wherein the filter structure comprises first filter elements (10) provided on the first surface (4) (of the first member (2)) and second filter elements (10) provided on the second surface (5) (of the second member (3)). It is particularly preferred that the filter elements (10) on the first and second member (3) interconnect. Such a filter structure can provide an improved filter resolution compared to a filter structure on one member.

Accordingly, in a preferred embodiment of the invention, in the assembled state of the nozzle assembly (1) the first filter elements (10) and the second filter elements (10) extend beyond the contact plane thereby forming a multiplicity of filter channels located between the first and second filter elements (10). An advantage is that the filter resolution of said filter is higher, than mechanically possible with a filter on only one member, due to the limitations of the generation of the filter elements (10) on the first and/or second member (3).

It is preferred in these embodiments that the lateral walls of the first filter elements (10) are not in contact with the lateral walls of the second filter elements (10). Such an arrangement allows to create filter channels with a smaller diameter than in a filter, wherein all elements are on one member. Accordingly, in some embodiments of the invention, the nozzle assembly (1) is a nozzle assembly as defined above, comprising filter elements (10) on the first and second member (3) wherein the cross-sectional diameter of the filter channels is smaller than the distance between neighbouring first or second filter elements (10).

The filter elements (10) on one member, may or may not make contact with the other member. It is preferred that the filter elements (10) of both members pass through the contact plane. It is further preferred that the filter elements (10) of the first member (2) are not in contact with the second member (3), in particular that the filter elements (10) are not in contact with the surface of the first member (2) and vice versa. Accordingly, in some embodiments the invention relates to a nozzle assembly (1) as defined above, wherein the first filter elements (10) are not in contact with the second surface (5) and wherein the second filter elements (10) are not in contact with the first surface (4).

In a further aspect of the invention, the invention relates to the use of a nozzle assembly (1) as defined above, in a nebulization device.

In a related aspect, the invention relates to a nebulization device comprising a nozzle assembly (1) as defined above.,

Said nebulization device can be any kind of nebulization device. The nozzle assembly (1) is suitable for a variety of nebulization devices. However it is preferred that the nebulization device is a medical nebulization device.

In particular preferred embodiments, said nebulization device is a medical inhalation device for the administration of a fluid medicament by inhalation to the lungs of a subject, and the medical inhalation device comprises a nozzle assembly (1) as defined by any embodiment above.

The invention is further described with reference to the figures.

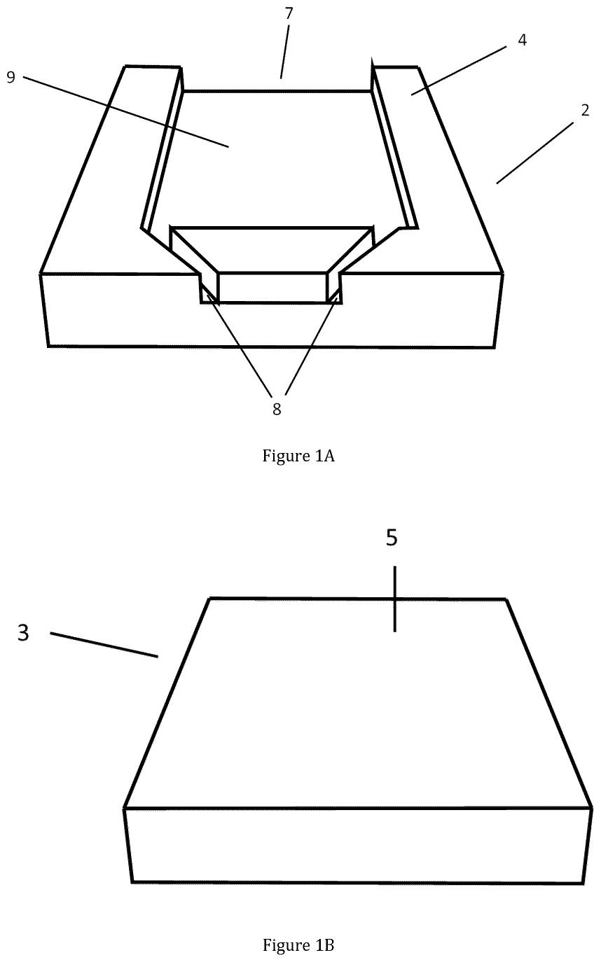

FIGS. 1A and 1B show a simple nozzle assembly (1) in accordance with the prior art. The nozzle assembly (1) comprises a first member (2), shown in FIG. 1A, with a first surface (4) and a second member (3), shown in FIG. 1B, with a second surface (5). In this embodiment according to the prior art the nozzle assembly does not comprise any positioning elements (6). The fluid inlet (7), fluid outlet (8) and connecting structure (9) are all realized on the first member (2) and the second member (3) essentially corresponds to a plate or a lid placed upon the first member (2).

In this embodiment, any displacement of the second member (3) relative to the first member (2) needs to be prevented using a third structure such as a nozzle holder or similar. However, this might not prevent small displacements, which depending on the design of the nozzle assembly can influence the effectiveness of the nozzle assembly.

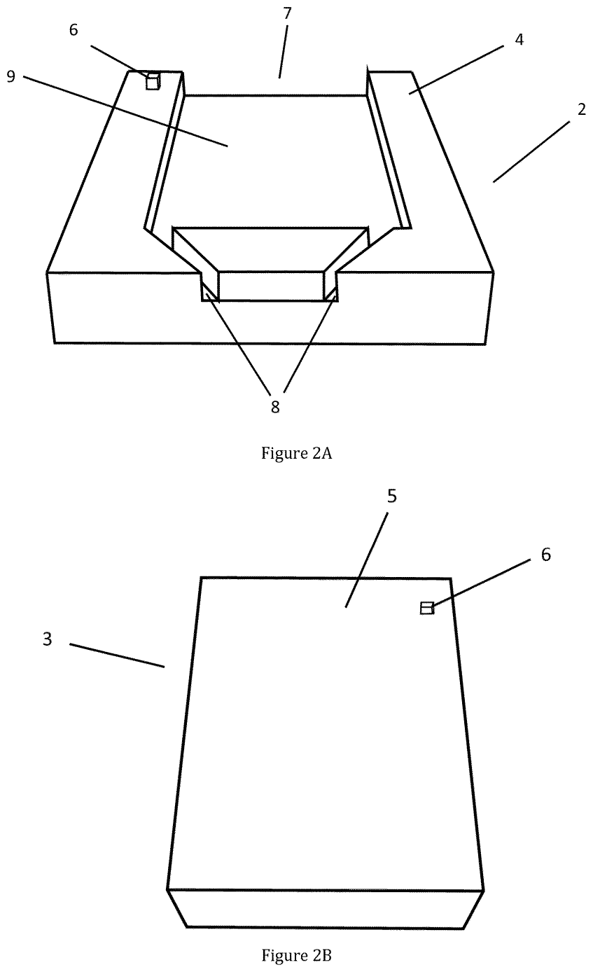

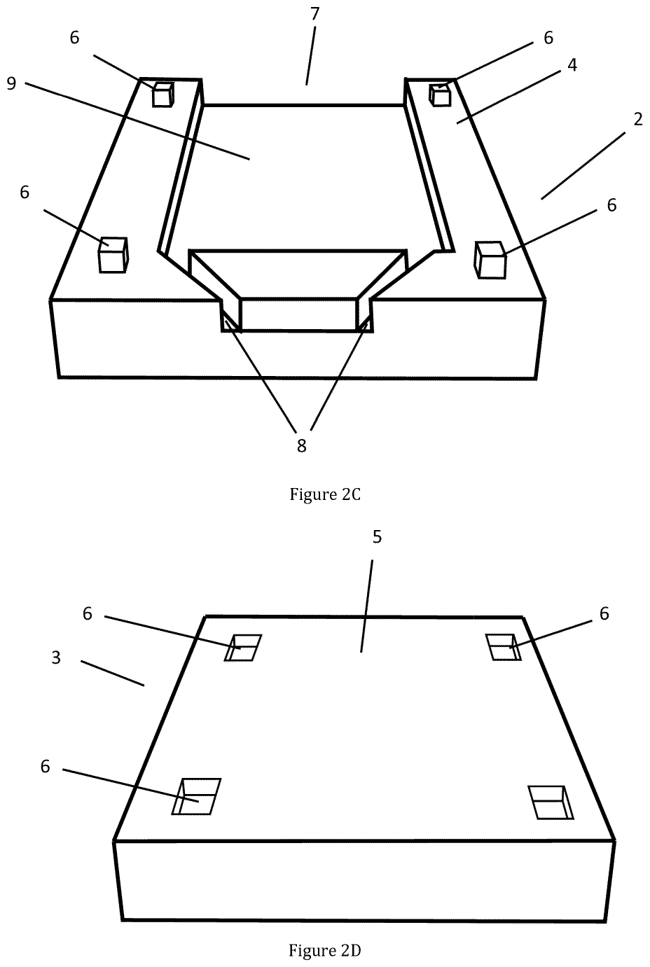

FIGS. 2A to 2D show two simple embodiments according to the present invention based on the simple nozzle assembly (1) shown in FIGS. 1A and B. FIGS. 2A and C show a first member (2) of a nozzle assembly, comprising a first surface (4) and a fluid inlet (7), fluid outlet (8) and connecting structure (9). Said first member (2) also comprises one (FIG. 2A) or four (FIG. 2C) positioning elements (6) on the first surface (4).

FIGS. 2B and 2D show the corresponding second member (3) comprising a second surface (5) and one (FIG. 2B) or four (FIG. 2D) positioning elements (6) corresponding to positioning elements (6) on the respective first member (2).

In case of the embodiment shown in FIGS. 2A and 2B the shape of the positioning element (6) is important, as a round positioning element (6) would allow for a rotation of the second member (3) and as such, the effectiveness of the prevention of displacement would be reduced. As soon as a plurality of corresponding positioning elements (6) is present on each member, a rotational displacement can be prevented.

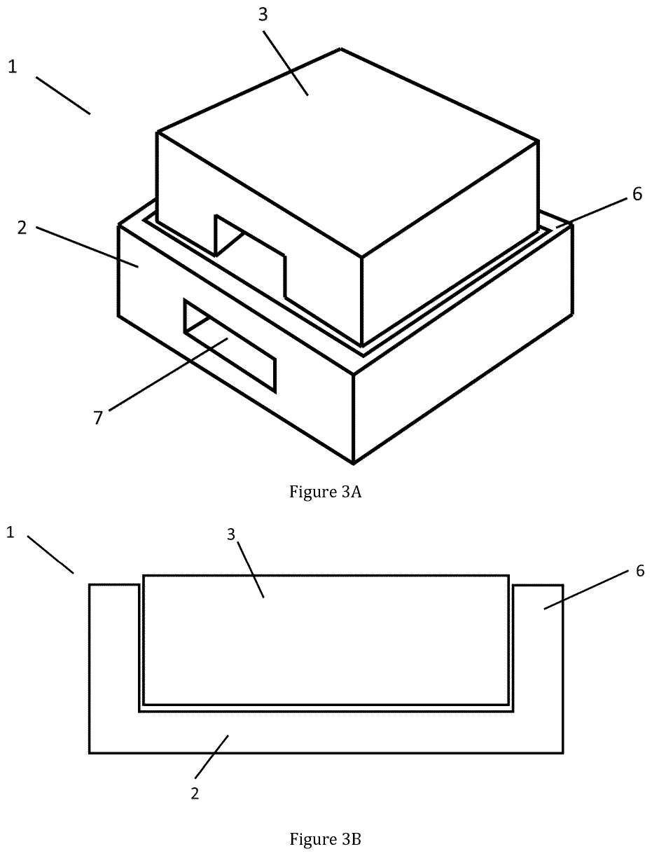

FIG. 3A shows a different embodiment of a nozzle assembly (1) according to the present invention. In this embodiment the positioning element (6) on the first member (2) is a higher wall, which fully encases the second member (3).

FIGS. 3B and 3C show a cross-section of said embodiment in two different variants. In FIG. 3B the second member (3) is positioned inside the “Walls” the positioning element(s) (6) of the first member (2). In this embodiment, vertical displacement of the second member (3) relative to the first member (2) is potentially possible, horizontal displacement is prevented. FIG. 3C shows an embodiment comprising additional protection to prevent horizontal and vertical displacement. In contrast to the embodiment of FIG. 3B the connection between the first member (2) and second member (3) is preferably irreversible.

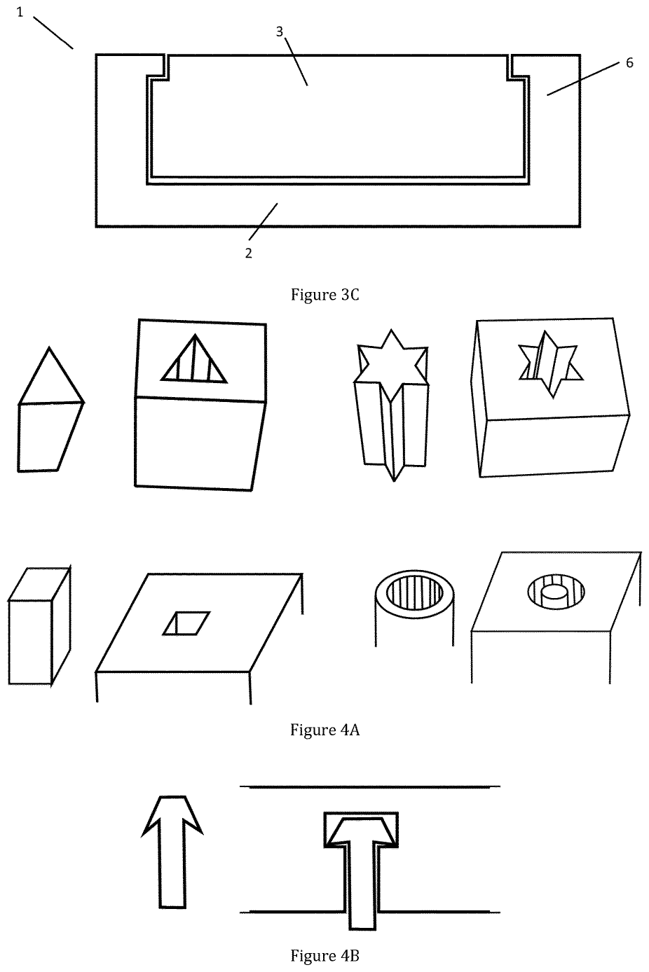

FIG. 4 A shows different possible embodiments for positioning elements (6) on the first member (2) and second member (3). These positioning elements (6) in combination allow for a reversible connection of the first and second member (3). These types of positioning elements (6) can be realized on any material for the first or second member (3).

FIG. 4B shows an example for a positioning element (6) for an irreversible connection. In this case, one of the positioning elements (6) is deformable and prevents a separation of the two members after the connection is completed. In this case, at least one of the first or send member has to be made at least partly of a more flexible material, such as plastic.

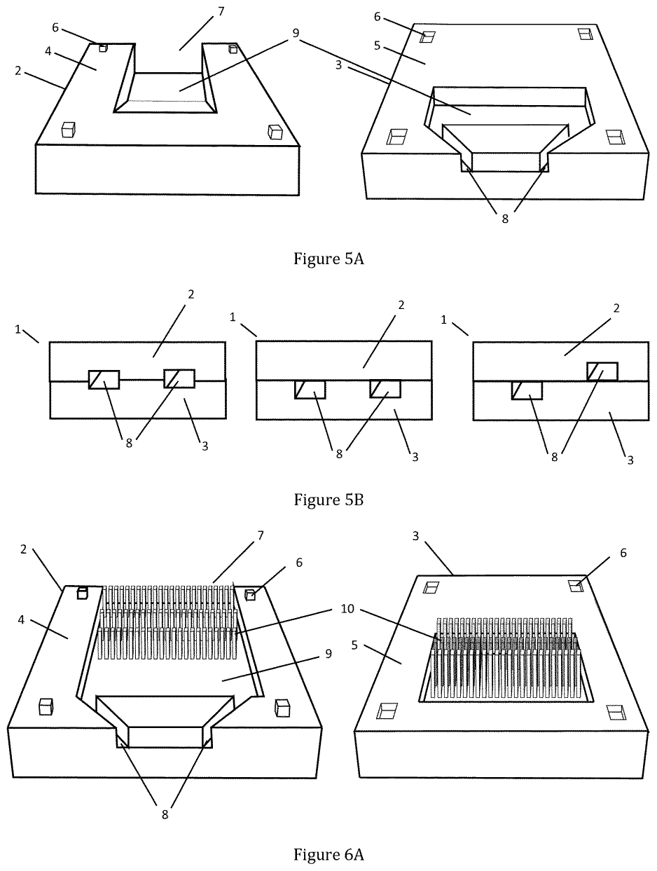

FIG. 5A shows a nozzle assembly (1) according to the invention, in which the connecting structure (9) is present on both the first and second member (2,3). In this specific embodiment, the fluid inlet (7) is present on the first member (2), while the fluid outlet (8) is on the second member (3). A connecting structure (9) is present on both members allowing for fluid transfer from inlet to outlet in the assembled state.

FIG. 5B shows different embodiments for the realization of two fluid outlets (8). Said outlets can be realized only one member, by both members or on each member.

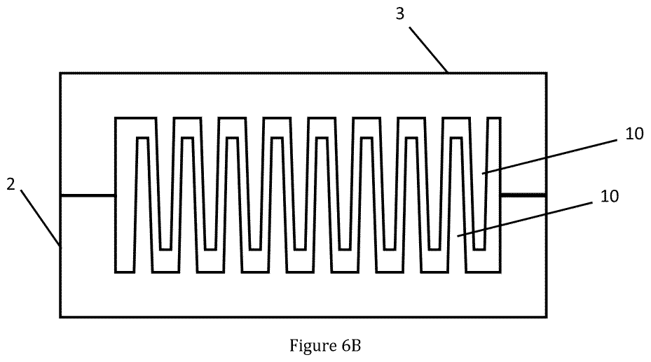

FIGS. 6A and 6B illustrate a further embodiment of the invention. FIG. 6 A illustrates a nozzle assembly (1) comprising a first (2) and second member (3), which each comprise filter elements (10). The combination of said filter elements allow for a higher filter resolution. This is exemplified in FIG. 6B which shows a cross-section through the nozzle assembly (1), in assembled state. In this embodiment the filter elements (10) on the first (2) and second (3) member interact to create an improved filtering resolution.

The invention further relates to the following numbered items.

1. A nozzle assembly (1) for the nebulization of a liquid, preferably of a medically active liquid, the nozzle assembly (1) comprising a first member (2) comprising a first surface (4) and a second member (3) comprising a second surface,
wherein the first member (2) comprises at least one positioning element (6) integrally formed on the first member (2), and
wherein in the assembled state of the nozzle assembly (1)
   the first surface (4) contacts the second surface, thereby defining a contact plane,
   the nozzle assembly (1) comprises a fluid inlet (7), a fluid outlet (8) and a connecting structure (9), all of which are in fluid communication forming a channel for the transport of the liquid in a downstream direction from the fluid inlet (7) via the connecting structure (9) to the fluid outlet (8),
   said channel is in contact with the contact plane and
   the at least one positioning element (6) formed on the first member (2) is in physical interaction with the second member (3) thereby preventing a displacement of the first member (2) relative to the second member (3).

2. The nozzle assembly (1) according to item 1, wherein the nozzle assembly (1) comprises a plurality of fluid outlets (8).

3. The nozzle assembly (1) according to items 1 or 2, wherein the second member (3) (also) comprises at least one positioning element (6) integrally formed on the second member (3), wherein in the assembled state of the nozzle assembly (1) the at least one positioning element (6) formed on the second member (3) is in physical interaction with the first member (2), thereby preventing a displacement of the first member (2) relative to the second member (3).

4. The nozzle assembly (1) according to item 3, wherein the positioning elements (6) on the first and second member (3) are corresponding male and female positioning elements (6).

5. The nozzle assembly (1) according to any one of the preceding items, wherein the first member (2) comprises a plurality of positioning elements (6).

6. The nozzle assembly (1) according to any one of the preceding items, wherein the second member (3) comprises a plurality of positioning elements (6).

7. The nozzle assembly (1) according to any one of the preceding items, wherein the positioning element (6) formed on the first member (2) is located on the first surface (4).

8. The nozzle assembly (1) according to any one of items 3 to 7, wherein the positioning element (6) formed on the second member (3) is located on the second surface.

9. The nozzle assembly (1) according to any one of the preceding items, wherein the positioning element (6) formed on the first member (2) is provided in the form of a projection (protuberance) projecting from the first surface (4).

10. The nozzle assembly (1) according to item 9, wherein, in the assembled state of the nozzle assembly (1), the projection projecting from the first surface (4) and extends beyond the contact plane of the nozzle assembly (1).

11. The nozzle assembly (1) according to any one of items 2 to 10, wherein the positioning element (6) formed on the second member (3) is provided in the form of an indentation (recess) formed in the second surface.

12. The nozzle assembly (1) according to any one of items 4 to 10, wherein the plurality of positioning elements (6) formed on the first member (2) comprises a positioning element (6) in the form of at a projection projecting from the first surface (4) and a positioning element (6) in the form of an indentation (recess) formed in the first surface (4).

13. The nozzle assembly (1) according to any one of items 6 to 12, wherein the plurality of positioning elements (6) formed on the second member (3) comprises one projection projecting from the second surface an indentation (recess) formed in the second surface.

14. The nozzle assembly (1) according to any one of items 2 to 12, wherein the positioning element (6) or the plurality of positioning elements (6) formed on the first surface (4) has the same position as the corresponding positioning element (6) or the plurality of positioning elements (6) formed on the second surface with regard to the contact plane.

15. The nozzle assembly (1) according to item 14, wherein the positioning element (6) or the plurality of positioning elements (6) formed on the first surface (4) mesh with the positioning element (6) or the plurality of positioning elements (6) formed on the second surface in the assembled state of the nozzle assembly (1).

16. The nozzle assembly (1) according to any one of the preceding items, wherein at least one of the first and second member (3) comprises a microcrystalline material.

17. The nozzle assembly (1) according to any one of the preceding items, wherein the first and the second member (3) each comprise a mono-crystalline material.

18. The nozzle assembly (1) according to any one of items 1 to 16, wherein one of the first and second members comprises a mono-crystalline material and the other one of the first and second members comprises glass.

19. The nozzle assembly (1) according to any one of items 16 to 18, wherein the mono-crystalline material is selected from silicon, germanium and gallium arsenide, preferably the mono-crystalline material is silicon.

20. The nozzle assembly (1) according to item 19, wherein the mono-crystalline material comprises a silicon wafer.

21. The nozzle assembly (1) according to any one of items 9 to 20, wherein the at least one positioning element (6) formed on the first member (2) in the form of a projection (protuberance) projecting from the first or second surface is provided in the form of a pin.

22. The nozzle assembly (1) according to any one of items 9 to 21, wherein the at least one positioning element (6) formed on the first or second member (3) in the form of an indentation (recess) formed in the second surface is provided in the form of a hole.

23. The nozzle assembly (1) according to any one of items 9 to 22, wherein the cross-sectional shape of the at least one positioning element (6) provided on the first surface (4) corresponds to the cross-sectional shape of the at least one positioning element provided on the second surface.

24. The nozzle assembly (1) according to any one of the preceding items, wherein the at least one positioning element (6), when provided in the form of a projection, has a height of from about 1 to about 100 μm, preferably of from about 3 to 30 μm as measured from the corresponding first or second surface.

25. The nozzle assembly (1) according to any one of the preceding items, wherein the at least one positioning element (6), when provided in the form of an indentation, has a depth of from about 1 to about 100 μm, preferably of from about 3 to 30 μm as measured from the corresponding first or second surface.

26. The nozzle assembly (1) according to any one of items 2 to 25, wherein the at least one protrusion is provided in a form of a linear or closed loop protrusion.

27. The nozzle assembly (1) according to item 26, wherein the linear protrusion is in the form of a straight or curved line or pattern of a plurality of linear protrusions.

28. The nozzle assembly (1) according to item 26, wherein the closed loop protrusion is provided in the form of a circular, oval, polygonal, star shaped or irregular shaped closed loop protrusion.

29. The nozzle assembly (1) according to any one of the preceding items, wherein the at least one positioning element (6) is adapted to prevent a horizontal displacement of the first member (2) relative to the second member (3) within the contact plane.

30. The nozzle assembly (1) according to any one of the preceding items, wherein the at least one positioning element (6) is adapted to prevent vertical displacement of the first member (2) relative to the second member (3) relative to the contact plane.

31. The nozzle assembly (1) according to any one of the preceding items, wherein the at least one positioning element (6) is adapted to generate a firm (preferably irreversible) connection between first member (2) and second member (3).

32. The nozzle assembly (1) according to any one of items 1 to 30, wherein the at least one positioning element (6) is adapted to generate a reversible connection between first member (2) and second member (3).

33. The nozzle assembly (1) according to any one of the preceding items, wherein in the assembled state of the nozzle assembly (1) the at least one positioning element (6) is not in contact with the liquid to be nebulized.

34. The nozzle assembly (1) according to any one of items 1 to 32, wherein in the assembled state of the nozzle assembly (1) the at least one positioning element (6) contacts the liquid to be nebulized.

35. The nozzle assembly (1) according to any one of the preceding items, wherein the liquid inlet, liquid outlet and the connecting structure (9) are provided on the first member (2).

36. The nozzle assembly (1) according to any one of the preceding items, wherein the at least one of the fluid inlet (7), fluid outlet (8) and connecting structure (9) which is provided in the form of a channel formed in at least one of the first surface (4) and the second surface has a depth of from about 1 μm to about 100 μm, preferably of from about 3 μm to about 30 μm.

37. The nozzle assembly (1) according to any one of the preceding items, wherein none of the fluid inlet (7), the fluid outlet (8) and the connecting structure (9) are provided on the second surface.

38. The nozzle assembly (1) according to any one of items 1 to 36, wherein the at least one of the liquid inlet and liquid outlet is provided on the first member (2) and wherein the connecting structure (9) is provided on the second member (3).

39. The nozzle assembly (1) according to any one of the preceding items, wherein the connecting structure (9) comprises at least one microfluidic element provided on the second surface of the second member (3).

40. The nozzle assembly (1) according to any one of the preceding items, wherein the at least one microfluidic element comprises a filter structure comprising at least one filter element.

41. The nozzle assembly (1) according to any one of the preceding claims, wherein the at least one microfluidic element and optionally at least one of the fluid inlet (7) and the fluid outlet (8) are provided by etching, embossing, scratching or cutting the corresponding element into the first and/or second surface (of the first and/or second member (3)).

42. The nozzle assembly (1) according to any one of the preceding items, wherein the fluid inlet (7), the fluid outlet (8) and the connecting structure (9) are located on the first surface (4) of the first member (2) and on the second surface of the second member (3).

43. The nozzle assembly (1) according to any one of the preceding items, wherein the fluid inlet (7), the fluid outlet (8) and the connecting structure (9) are provided in the form of mirror-image structures on the first and the second surface.

44. The nozzle assembly (1) according to any one of the preceding items, wherein the connecting structure (9) provided on at least one of the first and second surface comprise a bottom and lateral walls connecting the bottom with the first or the second surface.

45. The nozzle assembly (1) according to item 44, wherein the lateral walls are angled such that the inner diameter of the at least on of the fluid inlet (7), fluid outlet (8) and microfluidic structure widens from the bottom towards the first or second surface, correspondingly.

46. The nozzle assembly (1) according to any one of items 40 to 45, wherein the filter structure comprises first filter elements (10) provided on the first surface (4) (of the first member (2)) and second filter elements (10) provided on the second surface (of the second member (3)) and wherein in the assembled state of the nozzle assembly (1) the first filter elements (10) and the second filter elements (10) extend beyond the contact plane thereby forming a multiplicity of filter channels located between the first and second filter elements (10).

47. The nozzle assembly (1) according to item 46, wherein the lateral walls of the first filter elements (10) are not in contact with the lateral walls of the second filter elements (10).

48. The nozzle assembly (1) of item 46 or 47, wherein the first filter elements (10) are not in contact with the second surface and wherein the second filter elements (10) are not in contact with the first surface (4).

49. A medical inhalation device for the administration of a fluid medicament by inhalation to the lungs of a subject, wherein the medical inhalation device comprises a nozzle assembly (1) according to any one of the preceding items.

### LIST OF REFERENCE NUMBERS

1 Nozzle assembly
2 First member
3 Second member
4 First surface
5 Second surface
6 Positioning element
7 Fluid inlet
8 Fluid outlet
9 Connecting structure
10 Filter element

1. A nozzle assembly (1) for the nebulization of a liquid, preferably of a medically active liquid, the nozzle assembly (1) comprising a first member (2) comprising a first surface (4) and a second member (3) comprising a second surface,
wherein the first member (2) comprises at least one positioning element (6) integrally formed on the first member (2), and
wherein in the assembled state of the nozzle assembly (1)
the first surface (4) contacts the second surface, thereby defining a contact plane,
the nozzle assembly (1) comprises a fluid inlet (7), a fluid outlet (8) and a connecting structure (9), all of which are in fluid communication forming a channel for the transport of the liquid in a downstream direction from the fluid inlet (7) via the connecting structure (9) to the fluid outlet (8),
said channel is in contact with the contact plane and
the at least one positioning element (6) formed on the first member (2) is in physical interaction with the second member (3) thereby preventing a displacement of the first member (2) relative to the second member (3).

2. The nozzle assembly (1) according to claim 1, wherein the second member (3) (also) comprises at least one positioning element (6) integrally formed on the second member (3), wherein in the assembled state of the nozzle assembly (1) the at least one positioning element (6) formed on the second member (3) is in physical interaction with the first member (2), thereby preventing a displacement of the first member (2) relative to the second member (3).

3. The nozzle assembly (1) according to any one of the preceding claims, wherein the first and/or second member (2,3) comprises a plurality of positioning elements (6).

4. The nozzle assembly (1) according to any one of the preceding claims, wherein the positioning element (6) formed on the first member (2) is provided in the form of a projection (protuberance) projecting from the first surface (4).

5. The nozzle assembly (1) according to any one of claims 2 to 4, wherein the positioning element (6) formed on the second member (3) is provided in the form of an indentation (recess) formed in the second surface.

6. The nozzle assembly (1) according to any one of claims 4 or 5, wherein the plurality of positioning elements (6) formed on the first member (2) comprises a positioning element (6) in the form of at a projection projecting from the first surface (4) and a positioning element (6) in the form of an indentation (recess) formed in the first surface (4).

7. The nozzle assembly (1) according to any one of the preceding claims, wherein at least one of the first and second member (3) comprises a mono-crystalline material.

8. The nozzle assembly (1) according to any one of the preceding claims, wherein the connecting structure (9) comprises at least one microfluidic element.

9. The nozzle assembly (1) according to claim 8, wherein the at least one microfluidic element comprises a filter structure comprising at least one filter element.

10. The nozzle assembly (1) according to any one of the preceding claims, wherein at least one of the fluid inlet (7), the fluid outlet (8), the connecting structure (9) or a microfluidic element are provided in the form of mirror-image structures on the first and the second surface.

11. The nozzle assembly (1) according to any one of the preceding claims, wherein the connecting structure (9) provided on at least one of the first and second surface comprise a bottom and lateral walls connecting the bottom with the first or the second surface.

12. The nozzle assembly (1) according to any one of claims 8 to 11, wherein the at least one microfluidic element is a filter structure and wherein said filter structure comprises first filter elements (10) provided on the first surface (4) of the first member (2) and second filter elements (10) provided on the second surface of the second member (3) and wherein in the assembled state of the nozzle assembly (1) the first filter elements (10) and the second filter elements (10) extend beyond the contact plane thereby forming a multiplicity of filter channels located between the first and second filter elements (10).

13. The nozzle assembly (1) according to claim 12, wherein the lateral walls of the first filter elements (10) are not in contact with the lateral walls of the second filter elements (10).

14. The nozzle assembly (1) of claim 12 or 13, wherein the first filter elements (10) are not in contact with the second surface and wherein the second filter elements (10) are not in contact with the first surface (4).

15. A medical inhalation device for the administration of a fluid medicament by inhalation to the lungs of a subject, wherein the medical inhalation device comprises a nozzle assembly (1) according to any one of the preceding claims.
